(19) United States
(12) Patent Application Publication
(10) Pub. No.: US 2017/0107542 A1
(43) Pub. Date: Apr. 20, 2017

(54) TRANSGENIC PLANTS HAVING ALTERED EXPRESSION OF A XYLAN XYLOSYLTRANSFERASE AND METHODS OF USING SAME

(71) Applicant: UNIVERSITY OF GEORGIA RESEARCH FOUNDATION, INC., Athens, GA (US)

(72) Inventors: Debra Mohnen, Athens, GA (US); Ajaya Biswal, Athens, GA (US)

(21) Appl. No.: 15/299,123

(22) Filed: Oct. 20, 2016

Related U.S. Application Data
(60) Provisional application No. 62/243,814, filed on Oct. 20, 2015.

(51) Int. Cl. C12P 7/02

(52) U.S. Cl. CPC C12P 7/02; C12Y 204/02

(57) ABSTRACT

Provided herein are plants having altered expression of an IRX10 or an IRX10-L protein. Such plants have phenotypes that may include decreased recalcitrance, increased growth, decreased lignin content, or a combination thereof. Also provided herein are methods of making and using such plants.

### CROSS-REFERENCE TO RELATED APPLICATIONS

This application claims the benefit of U.S. Provisional Application Ser. No. 62/243,814, filed Oct. 20, 2015, which is incorporated by reference herein.

### GOVERNMENT FUNDING

The invention was made with government support under Grant No. DE-PS02-06ER64304, BioEnergy Science Center (BESC), awarded by the Department of Energy. The government has certain rights in this invention.

### SEQUENCE LISTING

This application contains a Sequence Listing electronically submitted via EFS-Web to the United States Patent and Trademark Office as an ASCII text file entitled “235_02550101_SeqList_ST25.txt” having a size of 32 kilobytes and created on Dec. 22, 2016. The information contained in the Sequence Listing is incorporated by reference herein.

### SUMMARY OF THE INVENTION

Provided herein are transgenic plants. In one embodiment, a transgenic plant has decreased expression of a coding region encoding an IRX10 or IRX10-L protein compared to a control plant. In one embodiment, the transgenic plant is not a SALK insertion line or a GABI-KAT insertion line. In one embodiment, the IRX10 or IRX10-L protein has at least 85% identity to SEQ ID NO:4, SEQ ID NO:6, SEQ ID NO:7, SEQ ID NO:8, SEQ ID NO:9, or SEQ ID NO:10. The transgenic plant can include a phenotype selected from decreased recalcitrance, increased growth, or the combination thereof. In one embodiment, the phenotype is decreased recalcitrance. The transgenic plant can be a monocot plant, such as a member of the family Poaceae. Examples of members of the family Poaceae is a member of a genus selected from Panicum, Zea, Oryza, Triticum, Hordeum, Sorghum, Avena, Secale, Fagopyrum, Digitara, and Chenopodium. In one embodiment, the transgenic plant is a member of the subfamily Panicoideae. The transgenic plant can be a woody plant, such as a member of the genus Populus or other woody plants including Pinus taeda, Salix sitchensis and Eucalyptus.

Also provided is a part of the transgenic plant, such as a leaf, a stem, a flower, an ovary, a fruit, a seed, or a callus; progeny of a transgenic plant, including a hybrid plant; biomass obtained from a transgenic plant described herein; and a pulp or biomass obtained from the transgenic plant.

Further provided are methods for using a transgenic plant described herein. In one embodiment, the method includes exposing material obtained from the plant to conditions suitable for the production of a metabolic product. In one embodiment, the exposing can include contacting the material with an ethanologenic microbe.

In one embodiment, the method includes processing a transgenic plant to result in pulp, wherein the transgenic plant includes decreased expression of a coding region encoding an IRX10 or IRX10-L protein compared to a control plant. The processing can include a physical pretreatment, a chemical pretreatment, or a combination thereof. The method can further include hydrolyzing the processed pulp.

In one embodiment, the method is for producing a metabolic product, and includes contacting under conditions suitable for the production of a metabolic product a microbe with a composition including a processed pulp obtained from a transgenic plant, wherein the transgenic plant includes decreased expression of a coding region encoding an IRX10 or IRX10-L protein compared to a control plant. The method can further include contacting the processed pulp with an ethanologenic microbe, such as a eukaryote.

In one embodiment, a method can further include obtaining a metabolic product. Examples of a metabolic product include an organic acid or an alcohol, such as ethanol, butanol, or a diol.

As used herein, the term “transgenic plant” refers to a plant that has been transformed to contain at least one modification to result in altered expression of a coding region. For example, a coding region in a plant may be modified to include a mutation to reduce transcription of the coding region or reduce activity of a protein encoded by the coding region, or alternatively to increase transcription of the coding region or increase activity of a protein encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. The term “transgenic plant” includes whole plants, plant parts (stems, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the term “wild-type” refers to a plant cell, seed, plant component, plant tissue, plant organ or whole plant that has not been genetically modified or treated in an experimental sense.

As used herein, the term “control plant” refers to a plant that is the same species as a transgenic plant, but has not been transformed with the same polynucleotide used to make the transgenic plant.

As used herein, the term “plant tissue” encompasses any portion of a plant, including plant cells, seed mucilage and root mucilage. Plant cells include suspension cultures, callus, embryos, meristematic regions, callus tissue, leaves, roots, shoots, gametophytes, sporophytes, pollen, seeds, and microspores. Plant tissues can be grown in liquid or solid culture, or in soil or suitable media in pots, greenhouses or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a protein encoded by the coding region.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a protein that may be encoded by the polynucleotide.

As used herein, “phenotype” refers to a distinguishing feature or characteristic of a plant which can be altered according to the present invention by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” refers to a modification of the natural nucleotide sequence of a coding region or an operably linked regulatory region in such a way that the protein encoded by the modified nucleic acid is altered structurally and/or functionally, or the coding region is expressed at a decreased level. Mutations may include, but are not limited to, mutations in a 5′ or 3′ untranslated region (UTR) or an exon, and such mutations may be a deletion, insertion, or point mutation to result in, for instance, a codon encoding a different amino acid or a stop to translation.

As used herein, a “target coding region” and “target coding sequence” refer to a specific coding region whose expression is inhibited by a polynucleotide described herein. As used herein, a “target mRNA” is an mRNA encoded by a target coding region.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, and protein are all included within the definition of protein and these terms are used interchangeably.

As used herein, a protein may be “structurally similar” to a reference protein if the amino acid sequence of the protein possesses a specified amount of sequence similarity and/or sequence identity compared to the reference protein. Thus, a protein may be “structurally similar” to a reference protein if, compared to the reference protein, it possesses a sufficient level of amino acid sequence identity, amino acid sequence similarity, or a combination thereof.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. A polynucleotide described herein may be isolated. An “isolated” polynucleotide is one that has been removed from its natural environment. Polynucleotides that are produced by recombinant, enzymatic, or chemical techniques are considered to be isolated and purified by definition, since they were never present in a natural environment.

As used herein, a polynucleotide may have “sequence similarity” to a reference polynucleotide if the nucleotide sequence of the polynucleotide possesses a specified amount of sequence identity compared to a reference polynucleotide. Thus, a polynucleotide may have “structural similarity” to a reference polynucleotide if, compared to the reference polynucleotide, it possesses a sufficient level of nucleotide sequence identity.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide.

“Hybridization” includes any process by which a strand of a nucleic acid sequence joins with a second nucleic acid sequence strand through base-pairing. Thus, strictly speaking, the term refers to the ability of a target sequence to bind to a test sequence, or vice-versa.

“Hybridization conditions” are typically classified by degree of “stringency” of the conditions under which hybridization is measured. The degree of stringency can be based, for example, on the calculated (estimated) melting temperature (Tm) of the nucleic acid sequence binding complex or probe. Calculation of Tm is known in the art (see Sambrook et al., 2001, Molecular Cloning, A Laboratory Manual, 3d Ed., Cold Spring Harbor Laboratory Press, Cold Spring Harbor, N.Y.). For example, “maximum stringency” typically occurs at about Tm −5° C. (5° below the Tm of the probe); “high stringency” at about 5-10° C. below the Tm; “intermediate stringency” at about 10-20° C. below the Tm of the probe; and “low stringency” at about 20-25° C. below the Tm. In general, hybridization conditions are carried out under high ionic strength conditions, for example, using 6×SSC or 6×SSPE. Under high stringency conditions, hybridization is followed by two washes with low salt solution, for example 0.5×SSC, at the calculated temperature. Under medium stringency conditions, hybridization is followed by two washes with medium salt solution, for example 2×SSC. Under low stringency conditions, hybridization is followed by two washes with high salt solution, for example 6×SSC. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, one will typically desire to employ relatively stringent conditions to form the hybrids, e.g., one will select relatively high temperature conditions. Hybridization conditions, including moderate stringency and high stringency, are provided in Sambrook et al., Molecular Cloning: A Laboratory Manual, Second Edition, Cold Spring Harbor Press (1989); Sambrook et al., Molecular Cloning, A Laboratory Manual, 3d Ed., Cold Spring Harbor Laboratory Press, Cold Spring Harbor, N.Y. (2001).

As used herein, “recalcitrance” refers to the natural resistance of plant cell walls to microbial and/or enzymatic and/or chemical deconstruction (see Fu et al., 2011, Proc. Natl. Acad. Sci. USA 108:3803-3808).

Conditions that are “suitable” for an event to occur, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows amino acid sequences of IRX10 and IRX10-L proteins and nucleotide sequences encoding IRX10 proteins.

FIG. 2 shows an amino acid alignment of eight IRX10 and IRX10-L proteins. At1g27440 (SEQ ID NO:1), AT5G61840 (SEQ ID NO:3), Pavirv00004837m, also referred to as Pavir.Ca01824 (SEQ ID NO:4), Pavirv00024113m (SEQ ID NO:6), Pavirv00013783m (SEQ ID NO:7), Pavirv00048255m (SEQ ID NO:8), Potri.001G068100 (SEQ ID NO:9), and Potri.012G109600 (SEQ ID NO:10)

FIG. 3A shows a phylogenetic tree of the GT47 Family of Arabidopsis thaliana (TAIR10) and Populus trichocarpa v3.0, Oryza sativa v7_{—JPI,} Panicum virgatum v1.1 (Phytozome 11.0) showing relationship between amino acid sequences, including the IRX10 and IRX10-L Clade. FIG. 3B shows a phylogenetic tree of the IRX10 and IRX10-L Clade from the GT47 Family. The tree was constructed by Neighbor-Joining method using MEGA6 (Tamura et al., 2011). Boxed proteins represent those encoded by genes used for the transgenic knockdown lines described herein.

FIG. 4 shows Gene model, vector map, relative expression, and growth phenotype of PdIRX10-L knockdown lines. FIG. 4A, Gene model of PtIRX10-L (Potri.012G109600) from Phytozome 11.0 Populus trichocarpa v3.0 with positions of the RNAi target sequence. Blue boxes indicate exons, lines indicate introns, and gray boxes are the 5′ and 3′ untranslated regions (UTRs). The RNAi target sequence was 135 by in the 3′UTR. FIG. 4B, Schematic presentation of IRX10-L (PtIRX10-L; Potri.012G109600) RNAi silencing construct. FIG. 4C, Phenotypes of wild type P. deltoides (left two plants), vector control (middle two plants), and PdIRX10-L KD lines (right two plants) of 3-month-old plants. FIG. 4D, Relative PtIRX10-L transcript level determined by quantitative RT-PCR analysis of 3-month-old plants. E to F, Height (E) and radial growth (F) of 3-month-old PdIRX10-L KD lines in comparison to wild type P. deltoides and vector controls. n=25 for wild type P. deltoides, n=15 for vector control lines and n=10-15 for PdIRX10-L KD lines. Data are average +/−SE.

FIG. 5 shows Gene model, vector map, relative expression, and growth phenotype of PdIRX10 knockdown lines. FIG. 5A, Gene model of PtIRX10 (Potri.001G068100) from Phytozome 11.0 Populus trichocarpa v3.0 with positions of the RNAi target sequence. Blue boxes indicate exons, lines indicate introns, and gray boxes are the 5′ and 3′ untranslated regions (UTRs). The RNAi target sequence was 172 by in the 5′UTR-to-2^{nd} Exon. FIG. 5B, Schematic presentation of IRX10 (PtIRX10-L; Potri.001G068100) RNAi silencing construct. FIG. 5C, Phenotypes of wild type and vector control P. deltoides (left two plants), and PdIRX10 KD lines (right two plants) of 3-month-old plants. FIG. 5D, Relative PtIRX10 transcript level determined by quantitative RT-PCR analysis of 3-month-old plants. E to F, Height (E) and radial growth (F) of 3-month-old PtIRX10 KD lines in comparison to wild type P. deltoides and vector controls. n=25 for wild type P. deltoides, n=15 for vector control lines and n=10-15 for PdIRX10-L KD lines. Data are average=/−SE.

FIG. 6 shows relative transcript abundance. WT-ST1, wild-type control; 196-1, 196-3, and 196-5, three unique transgenic switchgrass plants containing an IRX10 knock down (KD) mutation. A switchgrass ubiquitin gene was used as internal standard for normalization. The wild type control expression value (relative to the expression of ubiquitin) was taken as 100%.

FIG. 7 shows glucose release (top panel) and total sugar release (bottom panel). WT-ST1, wild-type control; 196-1, 196-3, and 196-5, three unique transgenic switchgrass plants containing an IRX10 KD mutation. Results are average+/−S.D. of two samples per line (* P<0.05).

FIG. 8 shows that saccharification yield is increased in the RNAi PdIRX10-L knockdown Populus deltoides lines. FIG. 8A to C, Glucose (A), xylose (B) and total sugar (C) release from wild type, vector control and PdIRX10-L lines. Mean±SE, Statistica 5.0 with one-way analysis of variance (ANOVA) followed by Tukey's multiple comparison test was used for statistical analysis. n=25 for wild type P. deltoides, 15 plants/clone for each vector control line; n=10-15 for PdIRX10-L lines.

FIG. 9 shows that saccharification yield is increased in the RNAi PdIRX10 knockdown Populus deltoides lines. FIG. 9A to C, Glucose (A), xylose (B) and total sugar (C) release from wild type, vector control and PdIRX10 lines. n=25 for wild type P. deltoides, 15 plants/clone for each vector control line; n=10-15 for PdIRX10-L lines. Mean±SE.

FIG. 10 shows increased plant height (top panel) and tiller number (bottom panel). WT-ST1, wild-type control; 196-1, 196-3, and 196-5, three unique transgenic switchgrass plants containing an IRX10 KD mutation. Data are average+/−S.D. of five plants per line (* P<0.05).

FIG. 11 shows mass yield. WT-ST1, wild-type control; 196-1, 196-3, and 196-5, three unique transgenic switchgrass plants containing an IRX10 KD mutation. Data are average+/−S.D. of four plants per line.

### DETAILED DESCRIPTION OF ILLUSTRATIVE EMBODIMENTS

### Polynucleotides and Proteins

The present invention includes, but is not limited to, a transgenic plant and cultured transgenic plant cells having an alteration in expression of a coding region encoding a xylan xylosyltransferase protein, having decreased IRX10/IRX10-L activity, or a combination thereof. A xylan xylosyltransferase protein is referred to herein as an IRX10 protein, and has biological activity that is referred to as xylan xylosyltransferase activity or IRX10/IRX10-L activity. The alterations in expression of an IRX10 or IRX10-L protein can include, but are not limited to, a decrease in expression of an active IRX10 or IRX10-L protein, expression of an inactive IRX10 or IRX10-L protein, expression of a IRX10 or IRX10-L protein that is altered to have decreased activity, the absence of detectable expression of a IRX10 or IRX10-L protein, or a decrease in IRX10 or IRX10-L activity. More than one protein can be altered in a cell or plant. In one embodiment, such modifications may be achieved by mutagenesis of a coding sequence encoding a GXMT protein.

IRX10 and IRX10-L are members of Carbohydrate-Active enZYmes (CAZY) GT Family 47 (Campbell et al. 1997, Biochem. J., 326:929-939; Coutinho et al., 2003, J. Mol. Biol., 328:307-317) and is co-transcriptionally regulated with secondary cell wall biosynthetic genes (Brown et al., 2005, Plant Cell 17:2281-95; Persson et al,. 2005, Proc Natl Acad Sci USA 102:8633-8). Heterologous expression in Pichia pastoris of purified IRX10 from Physcomitrella patens, Plantago ovata and Arabidopsis thaliana identified that IRX10 has the biological activity of elongating β-1,4-xlyotetrose to produce xylan, thereby identifying IRX10 as having xylan xylosytlranserase activity (Jensen et al., 2014, Plant J., 80(2):207-15 and Urbanowicz et al., 2014, Plant J., 80:197-206). The closely-related homolog of IRX10 in Arabidopsis, IRX10-L, has also been shown to have xylan synthase activity. IRX10-L has been named xylan synthase 1 (Urbanowicz et al., 2014, Plant J., 80:197-206).

As used herein, a protein having IRX10/IRX10-L activity means a protein catalyzes, under suitable conditions, synthesis of the xylan backbone by a β-1,4 xylose linkage. Whether a protein has IRX10/IRX10-L activity can be determined by in vitro assays. In one embodiment, an in vitro assay described by Jenson et al. (Jenson et al., 2014, Plant J., 80(2):207-15) can be used.

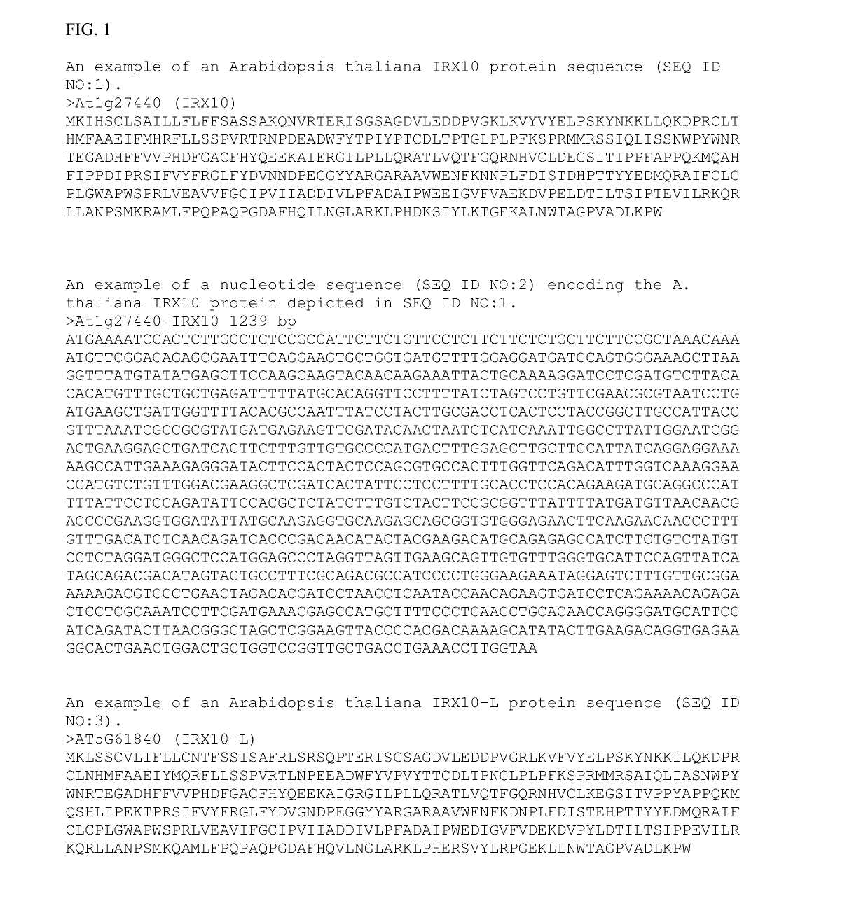

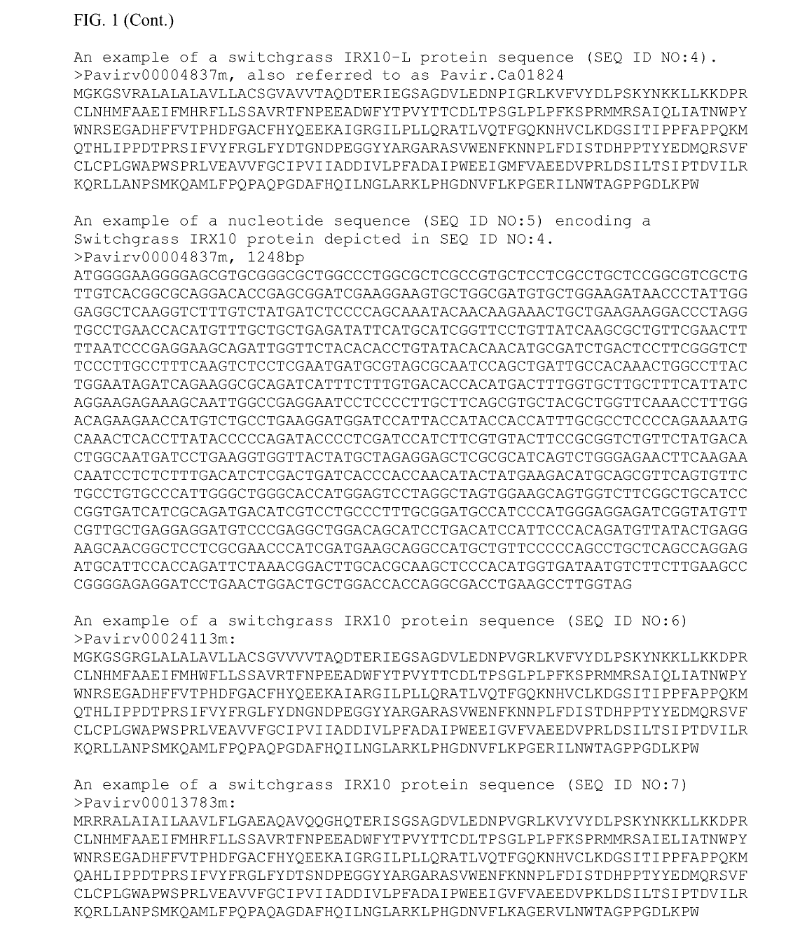

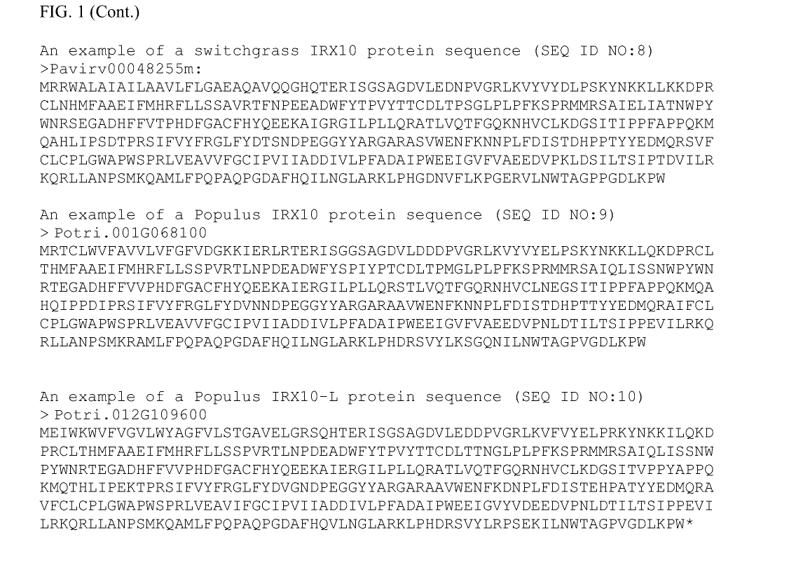

Examples of an IRX10 and IRX10-L proteins include SEQ ID NO:1 (an IRX10 from Arabidopsis thaliana), SEQ ID NO:3 (an IRX10L, a homolog of IRX10 in A. thaliana), SEQ ID NO:4 (IRX10-L protein from a switchgrass), SEQ ID NO:6, SEQ ID NO:7, and SEQ ID NO:8 (IRX10/IRX10-L proteins from a switchgrass), SEQ ID NO:9 (IRX10 from Populus trichocarpa) and SEQ ID NO:10 (IRX10-L from Populus trichocarpa) (FIG. 1). Other plants have homologs, including orthologs and paralogs, of these IRX10 and IRX10-L proteins. Other examples of IRX10 proteins include those that are structurally similar the amino acid sequence of SEQ ID NO:1, 3, 5, 6, 7, 8, 9, or 10.

Structural similarity of two proteins can be determined by aligning the residues of the two proteins (for example, a candidate protein and any appropriate reference protein such as SEQ ID NO:1, 5, 6, 7, 8, 9, or 10) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate protein is the protein being compared to the reference protein. A candidate protein may be isolated, for example, from a plant, or can be produced using recombinant techniques, or chemically or enzymatically synthesized. A candidate protein may be inferred from a nucleotide sequence present in the genome of a plant.

Unless modified as otherwise described herein, a pair-wise comparison analysis of amino acid sequences can be carried out using the Blastp program of the BLAST 2 search algorithm, as described by Tatiana et al., (1999, FEMS Microbiol Lett, 174:247-250), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, proteins may be compared using the BESTFIT algorithm in the GCG package (version 10.2, Madison, Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2.

Guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a protein sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

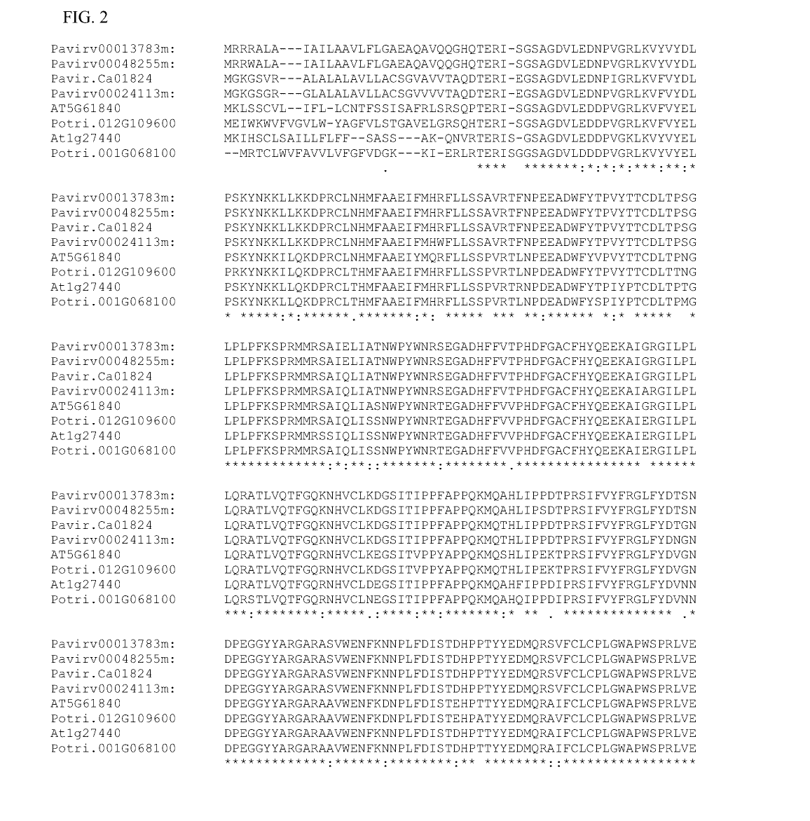

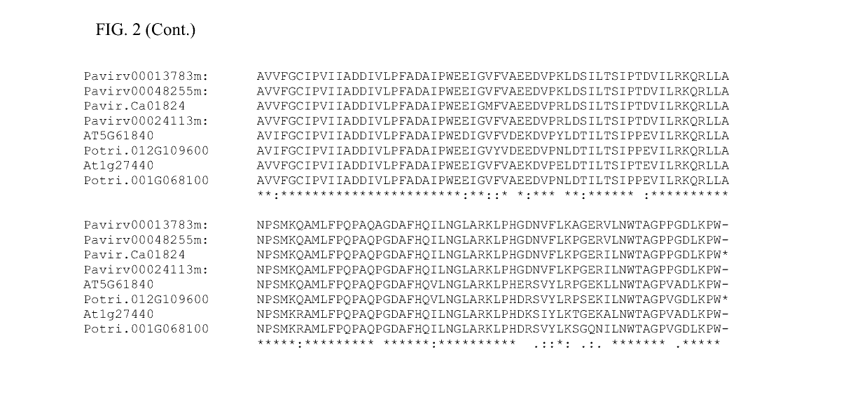

Guidance on how to modify the amino acid sequences of proteins disclosed herein is also provided at FIG. 2. FIG. 2 depicts a Clustl Omega amino acid alignment of IRX10 and IRX10-L proteins (Sievers et al., 2011, Molecular Systems Biology 7: 539, doi:10.1038/msb.2011.75; Goujon et al., 2010, Nucleic acids research 38 (Suppl 2):W695-9, doi:10.1093/nar/gkq313). In FIG. 2 an asterisk (*) indicates positions which have a single, fully conserved residue; a colon (:) indicates conservation between groups of strongly similar properties, roughly equivalent to scoring >0.5 in the Gonnet PAM 250 matrix; a period (.) indicates conservation between groups of weakly similar properties, roughly equivalent to scoring=<0.5 and >0 in the Gonnet PAM 250 matrix. By reference to this figure, the skilled person can predict which alterations to an amino acid sequence are likely to modify enzymatic activity, as well as which alterations are unlikely to modify enzymatic activity.

Thus, as used herein, a candidate protein useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to a reference amino acid sequence.

Alternatively, as used herein, a candidate protein useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Also provided herein are polynucleotides encoding an IRX10 protein. Examples of IRX10 polynucleotides are depicted at SEQ ID NOs:2 and 4. It should be understood that a polynucleotide encoding an IRX10 protein is not limited to a nucleotide sequence disclosed herein, but also includes the class of polynucleotides encoding the IRX10 protein as a result of the degeneracy of the genetic code. For example, the nucleotide sequence SEQ ID NO:5 is but one member of the class of nucleotide sequences encoding a protein having the amino acid sequence SEQ ID NO:4. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing the deoxyribonucleotide sequence with a ribonucleotide sequence and replacing each deoxythymidylate in the sequence with a ribouridylate nucleotide.

Other examples of IRX10 and IRX10-L proteins include those that are encoded by an IRX10 polynucleotide which has sequence similarity to SEQ ID NOs: 2 or 5. Sequence similarity of two polynucleotides can be determined by aligning the nucleotides of the two polynucleotides (for example, a candidate polynucleotide and SEQ ID NO: 2 or 5) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A candidate polynucleotide is the polynucleotide being compared to the reference polynucleotide. A candidate polynucleotide may be isolated, for example, from a plant, or can be produced using recombinant techniques, or chemically or enzymatically synthesized.

Unless modified as otherwise described herein, two nucleotide sequences can be compared using the Blastn program of the BLAST 2 search algorithm, as described by Tatiana et al., (1999, FEMS Microbiol Lett, 174:247-250), and available on the National Center for Biotechnology Information (NCBI) website. The default values for all BLAST 2 search parameters may be used.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to the reference nucleotide sequence.

Also provided herein are polynucleotides capable of hybridizing to SEQ ID NO: 2 or 5, or a complement thereof, and encoding an IRX10 or an IRX10-L protein. The hybridization conditions may be medium to high stringency. A maximum stringency hybridization can be used to identify or detect identical or near-identical polynucleotide sequences, while an intermediate or low stringency hybridization can be used to identify or detect polynucleotide sequence homologs.

IRX10 and IRX10-L are members of the CAZy glycosyltransferase family 47 (GT47) (Campbell et al., 1997, Biochem. J. 326:929-939; Coutinho et al., 2003, J. Mol. Biol. 328:307-317). The CAZy database describes the families of structurally-related catalytic and carbohydrate-binding modules (or functional domains) of enzymes that degrade, modify, or create glycosidic bonds (Cantarel et al., 2009, Nucleic Acids Res., 37:D233-238; Campbell et al., 1997, Biochem. J. 326:929-939; Coutinho et al., 2003, J. Mol. Biol. 328:307-317). The glycosyltransferase 47 (GT47) family (http://www.cazy.org) includes 39 members in Arabidopsis, 36 members in rice and 52 members in switchgrass. The genes irx10/irx10-L involved in xylan biosynthesis in Arabidopsis have been identified (Jensen et al., 2014, Plant J., 80:207-215; Urbanowicz et al., 2014, Plant J., 80:197-206) within the glycosyl transferase (GT) 47 family which also includes other GT47 family genes such as irx7(fra8)/irx7-L(F8H) (Zhong et al., 2005, Plant Cell 17:3390-408; Brown et al., 2007, Plant J., 52:1154-1168; Brown et al., 2009, Plant J., 57:732-746;Wu et al., 2010, Plant Physiol., 153:542-554; Wu et al., 2009, Plant J., 57:718-731).

The present invention also provides methods of using IRX10 and IRX10-L proteins and polynucleotides encoding IRX10 and IRX10-L proteins. The present invention includes methods for altering expression of plant IRX10 and IRX10-L coding regions for purposes including, but not limited to (i) investigating function of biosynthesis of xylan and pectin, (ii) investigating the function and structure of the plant cell walls including polysaccharides and proteoglycans, (iii) effecting a change in plant phenotype, and (iv) using plants having an altered phenotype.

The present invention includes methods for altering the expression of a coding region present in the genome of a plant and encoding an IRX10 or an IRX10-L protein. Thus, for example, the invention includes altering expression of an IRX10 or an IRX10-L coding region present in the genome of a wild-type plant. In one embodiment, expression of more than one IRX10 or IRX10-L coding region present in the genome of a wild-type plant is altered. In one embodiment a wild-type plant is a member of the family Poaceae, such as a member of the genus Panicum, including P. virgatum.

Techniques which can be used in accordance with the present invention to alter expression of an IRX10 coding region, include, but are not limited to: (i) over-expression of the coding region, (ii) decreasing expression of the coding region (e.g., disrupting a coding region's transcript, such as disrupting a coding region's mRNA transcript; disrupting the function of a protein encoded by a coding region, or disrupting the coding region itself) or (iii) modifying the timing of expression of the coding region by placing it under the control of a non-native promoter. The use of antisense RNAs, ribozymes, double-stranded RNA interference (dsRNAi), and gene knockouts are valuable techniques for discovering the functional effects of a coding region and for generating plants with a phenotype that is different from a wild-type plant of the same species.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726, and Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Small, 2007, Curr. Opin. Biotechnol., 18:148-153; McGinnis, 1010, Brief. Funct. Genomics, 9(2): 111-117. A non-limiting example of a nucleotide sequence that can be used to inhibit IRX10 expression in a switchgrass is:

(SEQ ID NO: 11)
TCATGCATCGGTTCCTGTTATCAAGCGCTGTTCGAACTTTTAATCCCGAG
GAAGCAGATTGGTTCTACACACCTGTATACACAACATGCGATCTGACTCC
TTCGGGTCTTCCCTTGCCTTTCAAGTCTCCTCGAATGATGCGTAGCGCAA
TCCAGCTGATTGCCACAAACTGGCCTTACTGGAATAGATCAGAAGGCGCA
GATCATTTCTTTGTGACACCACATGACTTTGGTGCTTGCTTTCATTATCA
GGAAGAGAAAGCAATTGGCCGAGGAATCCTCCCCTTGCTTCAGCGTGCTA
CGCTGGTTCAAACCTTTGGACAGAAGAACCATGTCTGCCTGAAGGATGGA
TCCATTACCATACCACCATTTGCGCCTCCCCAGAAAATGCAAACTCACCT
TATACCCCCAGATACCCCTCGATCCATCTTCGTGTACTTCCGCGGTCTGT
TCTATGACACTGGCAATGATCCTGAAGGTGGT.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the protein encoded by the coding region. For instance, mutations in a coding region and/or an operably linked regulatory region may be made by deleting, substituting, or adding a nucleotide(s). The use of T-DNA based inactivation is discussed, for example, in Azpiroz-Leehan et al. (1997, Trends in Genetics, 13:152-156). Disruption of a coding region may also be accomplished using methods that include transposons, homologous recombination, and the like.

Altering expression of an IRX10 coding region may be accomplished by using a portion of a polynucleotide described herein. In one embodiment, a polynucleotide for altering expression of an IRX10 coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of an IRX10 coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of an IRX10 coding region in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a IRX10 coding region in a plant cell includes substantially all of a coding region, or in some cases, an entire coding region. An antisense strand is substantially complementary, preferably, complementary, to a target coding region or a target mRNA. As used herein, the term “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode an antisense RNA molecule or a molecule useful in RNAi. Promoters useful in the invention include constitutive promoters, inducible promoters, and/or tissue preferred promoters for expression of a polynucleotide in a particular tissue or intracellular environment, examples of which are known to one of ordinary skill in the art.

Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

Another example of a regulatory region is a transcription terminator. Suitable transcription terminators are known in the art and include, for instance, a stretch of 5 consecutive thymidine nucleotides.

Thus, in one embodiment a polynucleotide that is operably linked to a regulatory sequence may be in an “antisense” orientation, the transcription of which produces a polynucleotide which can form secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual, Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli or Agrobacterium tumefaciens. Preferably the vector is a plasmid. In some embodiments, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli or A. tumefaciens.

A selection marker is useful in identifying and selecting transformed plant cells or plants. Examples of such markers include, but are not limited to, a neomycin phosphotransferase (NPTII) gene (Potrykus et al., 1985, Mol. Gen. Genet., 199:183-188), which confers kanamycin resistance, and a hygromycin B phosphotransfease (HPTII) gene (Kaster, et al, 1983, Nuc. Acid. Res. 19: 6895-6911). Cells expressing the NPTII gene can be selected using an appropriate antibiotic such as kanamycin or G418. The HPTII gene encodes a hygromycin-B 4-O-kinase that confers hygromycin B resistance. Cells expressing HPTII gene can be selected using the antibiotic of hygromycin B (Kaster, et al, 1983, Nuc. Acid. Res. 19: 6895-6911, Blochlinger and Diggelmann, 1984, Mol. Cell. Biol. 4 (12): 2929-2931). Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide described herein in a cell, and the polynucleotide may then be isolated from the cell.

### Host Cells, Plants, and Transgenic Plants

The invention also provides host cells having altered expression of a coding region encoding a xylan xylosyltransferase protein, having decreased IRX10 activity, having decreased IRX10-L activity, or a combination thereof. In one embodiment, a host cell includes decreased IRX10 activity or decreased IRX10-L activity compared to a control cell, wherein the host cell is not a cell from the SALK insertion line, e.g., SALK046368 or from the GABI-Kat insertion line, e.g., Gabi 179G11. In one embodiment, a host cell includes decreased expression of a coding region encoding an IRX10 protein or an IRX10-L protein compared to a control cell, wherein the host cell is not a cell from the SALK insertion line, e.g., SALK046368 or from the GABI-Kat insertion line, e.g., Gabi 179G11. A host cell may be homozygous or heterozygous for a modification that results in altered expression of a coding region. More than one IRX10 or IRX10-L protein can be altered in a host cell. As used herein, a host cell includes the cell into which a polynucleotide described herein was introduced (a recombinant host cell), and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

The present invention further provides transgenic plants having altered expression of a coding region encoding IRX10 or IRX10-L. In one embodiment, a transgenic plant includes decreased IRX10 activity or IRX10-L activity compared to a control plant, wherein the transgenic plant is not a plant line from the SALK insertion line, e.g., SALK046368 or from the GABI-Kat insertion line, e.g., Gabi 179G11. In one embodiment, a transgenic plant includes decreased expression of a coding region encoding an IRX10 protein compared to a control plant, wherein the transgenic plant is not a plant line from the SALK insertion line, e.g., SALK046368 or from the GABI-Kat insertion line, e.g., Gabi 179G11. A transgenic plant may be homozygous or heterozygous for a modification that results in altered expression of a coding region. More than one IRX10 protein or IRX10-L protein can be altered in a cell or plant.

An IRX10 or IRX10-L protein that is altered to have decreased activity may be decreased by at least 10%, at least 20%, at least 25%, at least 30%, at least 35%, at least 40%, at least 45%, at least 50%, at least 55%, at least 60%, at least 65%, at least 70%, at least 75%, at least 80%, or at least 90% compared to the activity of an IRX10 or IRX10-L in a control plant or control cell. In one embodiment, host cell or a transgenic plant may have an absence of detectable activity of an IRX10 or IRX10-L (also referred to as a knockout or KO). In one embodiment, the host cell or a transgenic plant may have a decrease in IRX10 or IRX10-L expression. The IRX10 or IRX10-L expression in a host cell or a transgenic plant may be decreased by at least 10%, at least 20%, at least 25%, at least 30%, at least 35%, at least 40%, at least 45%, at least 50%, at least 55%, at least 60%, at least 65%, at least 70%, at least 75%, at least 80%, or at least 90% compared to the IRX10 or IRX10-L expression in a control plant. In one embodiment, host cell or a transgenic plant may have an absence of detectable expression of an IRX10 or IRX10-L. A transgenic plant having no detectable IRX10 or IRX10-L activity or expressin is also referred to as a knockout (KO). A transgenic plant having reduced but detectable IRX10 or IRX10-L activity or expressin is also referred to as a knockdown (KD).

In one embodiment, a host cell or transgenic plant has decreased expression of an IRX10 or IRX10-L protein, where the decrease is measured as a reduction in the transcript encoding the IRX10 or IRX10-L protein. The decrease in transcript level can be at least 35% or at least 40% to no greater than 50% to no greater than 55%. This range of reduction in transcript level has been identified as resulting in transgenic plants that have reduced recalcitrance and increased growth.

The present invention also includes natural variants of plants, where the natural variants have increased or decreased expression of IRX10 or IRX10-L proteins. In one embodiment, IRX10 or IRX10-L expression is decreased. The change in IRX10 or IRX10-L expression is relative to the level of expression of the IRX10 or IRX10-L protein in a natural population of the same species of plant. Natural populations include natural variants, and at a low level, extreme variants (Studer et al., 2011, 108:6300-6305). The level of expression of IRX10 or IRX10-L protein in an extreme variant may vary from the average level of expression of the IRX10 or IRX10-L protein in a natural population by at least 5%, at least 10%, at least 15%, at least 20%, or at least 25%. The average level of expression of the IRX10 or IRX10-L protein in a natural population may be determined by using at least 50 randomly chosen plants of the same species as the putative extreme variant.

A plant may be an angiosperm or a gymnosperm. The polynucleotides described herein may be used to transform a variety of plants, both monocotyledonous (e.g grasses, corn, grains, oat, wheat, barley), dicotyledonous (e.g., Arabidopsis, tobacco, legumes, alfalfa, oaks, eucalyptus, maple, poplar, aspen, cottonwood), and Gymnosperms (e.g., Scots pine, white spruce, and larch).

A plant may be switchgrass (e.g., a member of the genus Panicum, such as P. virgatum), turfgrass, wheat, maize, rice, sugar beet, potato, tomato, lettuce, carrot, strawberry, cassava, sweet potato, geranium, soybean, and various types of woody plants. Woody plants include trees such as palm oak, pine, maple, fir, apple, fig, plum acacia, poplar, aspen, cottonwood, and willow. Woody plants also include rose and grape vines.

In one embodiment, a plant is a member of the family Poaceae. For instance, a plant may be a member of a genus selected from Panicum, Zea, Oryza, Triticum, Hordeum, Sorghum, Avena, Secale, Fagopyrum, Digitara, and Chenopodium. For instance, a plant may be a member of the subfamily Panicoideae.

In one embodiment, the plants are woody plants, which are trees or shrubs whose stems live for a number of years and increase in diameter each year by the addition of woody tissue, such as members of the family Salicaceae, such as Populus spp. (e.g., Populus trichocarpa, Populus deltoides), pine, and Eucalyptus spp. In another embodiment the plants are herbaceous plants and forage crops such as alfalfa and switchgrass. We expect IRX10 to affect secondary cell wall formation. However, it may also affect primary wall formation as in T-DNA transformed tobacco callus (Wu et al, 2009; Iwai et al., 2002; Iwai et al., 2006).

Also included in the present invention is the biomass (e.g., wood, agricultural waste), and the pulp derived from the plants described herein.

Transformation of a plant with a polynucleotide described herein to result in decreased IRX10 or IRX10-L activity, expression, or the combination thereof, may yield a phenotype including, but not limited to any one or more of changes in root growth, height, stem width, yield, lignin quality, lignin structure, amount of lignin, pectin structure, hemicellulose structure, glycoconjugate structure, wood composition, wood strength, cellulose polymerization, fiber dimensions, cell wall composition (such as cell wall polysaccharide content), rate of wood formation, rate of growth, amount of inflorescence development or production, leaf shape, wood flexibility, and wood strength. In one embodiment a phenotype is increased glucose yield per gram biomass, increased total sugar yield per gram biomass, decreased recalcitrance, increased saccharification efficiency, increased growth (such as increased height, increased tiller number, increased dry weight/plant, increased aerial dry weight/plant, or the combination thereof) compared to a control plant. Methods for measuring each of these characteristics are known to the skilled person and routine. For instance, methods for measuring recalcitrance are routine and include, but are not limited to, measuring changes in the extractability of carbohydrates, where an increase in extractability suggests a more loosely held together wall, and thus, decreased recalcitrance. Another test for measuring changes in recalcitrance uses microbes as described in Mohnen et al. (WO 2011/130666).

Other phenotypes present in a transgenic plant described herein may include yielding biomass with reduced recalcitrance and from which sugars can be released more efficiently for use in biofuel and biomaterial production, yielding biomass which is more easily deconstructed and allows more efficient use of wall structural polymers and components, and yielding biomass that will be less costly to refine for recovery of sugars and biomaterials.

Phenotype can be assessed by any suitable means. The plants may be evaluated based on their general morphology. Transgenic plants can be observed with the naked eye, can be weighed and their height measured. The plant can be examined by isolating individual layers of plant tissue, e.g. phloem and cambium, and also by examining meristematic cells, early expansion tissue, late expansion tissue, and at secondary wall formation, late cell maturation and primary wall formation stages. The plants also can be assessed using microscopic analysis or chemical analysis.

Microscopic analysis includes examining cell types, stage of development, and stain uptake by tissues and cells. Fiber morphology, such as fiber wall thickness may be observed using, for example, microscopic transmission ellipsometry (Ye and Sundstrom, 1977, Tappi J., 80:181). Wood strength and density in wet wood and standing trees can be determined by measuring the visible and near infrared spectral data in conjunction with multivariate analysis (Gabor, U.S. Pat. No. 6,525,319). Lumen size can be measured using scanning electron microscopy. Lignin structure and chemical properties, (such as cell wall properties) can be observed using nuclear magnetic resonance spectroscopy, chemical derivatization, mass spectrometry, diverse microscopies, colorimetric assays, and glycome profiling.

The biochemical characteristics of lignin, cellulose, carbohydrates and other plant extracts can be evaluated by standard analytical methods including spectrophotometry, fluorescence spectroscopy, HPLC, mass spectroscopy, molecular beam mass spectroscopy, near infrared spectroscopy, nuclear magnetic resonance spectroscopy, and tissue staining methods.

One method that can be used to evaluate the phenotype of a transgenic plant is glycome profiling. Glycome profiling gives information about the presence of carbohydrate structures in plant cell walls, including changes in the extractability of carbohydrates from cell walls (Zhu et al., 2010, Mol. Plant, 3:818-833; Pattathil et al., 2010, Plant Physiol., 153:514-525), the latter providing information about larger scale changes in wall structure. In one embodiment the change is an increase of one or more carbohydrates in an extracted fraction compared to a control plant. Examples of solvents useful for evaluating the extractability of carbohydrates include, but are not limited to, oxalate, carbonate, KOH (e.g., 1M and 4M), and chlorite. Diverse plant glycan-directed monoclonal antibodies are available from, for instance, CarboSource Services (Athens, Ga.), and PlantProbes (Leeds, UK).

### Methods for Making

Transgenic plants described herein may be produced using routine methods. Methods for transformation and regeneration are known to the skilled person. Transformation of a plant cell with a polynucleotide described herein to yield a recombinant host cell may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, chloroplast transformation and using CRISPR/Cas9 technology.

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This may be accomplished by, for instance, PEG or electroporation mediated-uptake, particle bombardment-mediated delivery, or microinjection. In each case the transformed cells may be regenerated to whole plants using standard techniques known in the art.

Techniques for the transformation of monocotyledon species include direct gene transfer into protoplasts using PEG or electroporation techniques, particle bombardment into callus tissue or organized structures, CRISPR/Cas9 technology, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

Natural variants as described herein may be identified using known and routine techniques.

### Methods of Use

Provided herein are methods for using the plants described herein. In one embodiment, the methods include producing a metabolic product. A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus the invention includes methods for processing a transgenic plant to result in a pulp, methods for hydrolyzing a pulp that contain cells from a transgenic plant, and methods for producing a metabolic product from a pulp.

There are numerous methods or combinations of methods known in the art and routinely used to process plants. The result of processing a plant is a pulp. As used herein, “pulp” refers to processed plant material. Plant material, which can be any part of a plant, may be processed by any means, including mechanical, chemical, biological, or a combination thereof. Mechanical pretreatment breaks down the size of plant material. Biomass from agricultural residues is often mechanically broken up during harvesting. Other types of mechanical processing include milling or aqueous/steam processing. Chipping or grinding may be used to typically produce particles between 0.2 and 30 mm in size. Methods used for plant materials may include intense physical pretreatments such as steam explosion and other such treatments (Peterson et al., U.S. Patent Application 20090093028). The most common chemical pretreatment methods used for plant materials include dilute acid, alkaline, organic solvent, ammonia, sulfur dioxide, carbon dioxide or other chemicals to make the biomass more available to enzymes. Biological pretreatments are sometimes used in combination with chemical treatments to solubilize lignin in order to make cellulose more accessible to hydrolysis and fermentation. In one embodiment, a method for using transgenic plants described herein includes processing plant material to result in a pulp. In one embodiment, transgenic plants described herein, such as those with reduced recalcitrance and/or decreased lignification, are expected to require less processing than a control plant. The conditions described below for different types of processing are for a control plant, and the use of a plant as described herein is expected to require less severe conditions.

Steam explosion is a common method for pretreatment of plant biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes degradation of cell wall complex carbohydrates and lignin transformation. Addition of H₂SO₄, SO₂, or CO₂ to the steam explosion reaction can improve subsequent cellulose hydrolysis (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of plant biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment. Hot water, for example 140° C. or 160° C. or 180° C. can also be used as a pretreatment of plant biomass (Studer et al, 2011, Proc. Natl. Acad. Sci., U.S.A., 108:6300-6305).

Methods for hydrolyzing a pulp may include enzymatic hydrolysis, oxidation or lyase action. Enzymatic hydrolysis of processed biomass includes the use of cellulases. Some of the pretreatment processes described above include hydrolysis of complex carbohydrates, such as hemicellulose and cellulose, to monomer sugars. Pectinases may also be used to breakdown the pectic complex carbohydrates and proteoglycans in biomass (Atmodjo et al., 2013). Others, such as organosolv, prepare the substrates so that they will be susceptible to hydrolysis. This hydrolysis step can in fact be part of the fermentation process if some methods, such as simultaneous saccharification and fermentation (SSF), are used. Otherwise, the pretreatment may be followed by enzymatic hydrolysis with cellulases.

A cellulase may be any enzyme involved in the degradation of the complex carbohydrates in plant cell walls to fermentable sugars, such as glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, e.g., hydrolyze cellulose, either in the acid, neutral, or alkaline pH-range.

A cellulase may be of fungal or bacterial origin, which may be obtainable or isolated from microorganisms which are known to be capable of producing cellulolytic enzymes. Useful cellulases may be produced by fermentation of the above-noted microbial strains on a nutrient medium containing suitable carbon and nitrogen sources and inorganic salts, using procedures known in the art.

Examples of cellulases suitable for use in the present invention include, but are not limited to, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH).

The hydrolysis/fermentation of plant material may, and typically does, require addition of cellulases (e.g., cellulases available from Novozymes A/S). Typically, cellulase enzymes may be added in amounts effective from 5 to 35 filter paper units of activity per gram of substrate, or, for instance, 0.001% to 5.0% wt. of solids. The amount of cellulases appropriate for the hydrolysis may be decreased by using a transgenic plant described herein. The amount of cellulases (e.g., cellulases available from Novozymes A/S) required for hydrolysis of the pretreated plant biomass may be decreased by at least 5%, at least 10%, at least 15%, at least 20%, at least 25%, at least 30%, or at least 40% compared to the amount of cellulases required for hydrolysis of a control plant. This decreased need for cellulases can result in a significant decrease in costs associated with producing metabolic products from plant materials.

The steps following pretreatment, e.g., hydrolysis and fermentation, can be performed separately or simultaneously. Conventional methods used to process the plant material in accordance with the methods disclosed herein are well understood to those skilled in the art. Detailed discussion of methods and protocols for the production of ethanol from biomass are reviewed in Wyman (1999, Annu. Rev. Energy Environ., 24:189-226), Gong et al. (1999, Adv. Biochem. Engng. Biotech., 65: 207-241), Sun and Cheng (2002, Bioresource Technol., 83:1-11), and Olsson and Hahn-Hagerdal (1996, Enzyme and Microb. Technol., 18:312-331). The methods of the present invention may be implemented using any conventional biomass processing apparatus (also referred to herein as a bioreactor) configured to operate in accordance with the invention. Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Appl. Biochem. Biotechnol., 56: 141-153). Smaller scale fermentations may be conducted using, for instance, a flask.

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC). The fermentation can be carried out by batch fermentation or by fed-batch fermentation.

SHF uses separate process steps to first enzymatically hydrolyze plant material to glucose (and other sugars) and then ferment glucose (and other sugars) to ethanol. In SSF, the enzymatic hydrolysis of plant material and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the cofermentation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature than the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

The final step may be recovery of the metabolic product. Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

Transgenic plants described herein may also be used as a feedstock for livestock. Plants with reduced recalcitrance are expected to be more easily digested by an animal and more efficiently converted into animal mass. Accordingly, the present invention includes methods for using a transgenic plant as a source for a feedstock, and includes a feedstock that has plant material from a transgenic plant as one of its components.

Transgenic plants described herein may also be used for production of biomaterials. Modified wall structure and increased ease of extraction may make the isolation and generation of biomass-related products easier and more cost effective. Accordingly, the present invention includes methods for using a transgenic plant as a source of modified biomass with more facile biomass extraction and mechanical properties and includes a feedstock that has plant material from a transgenic plant as one of its components.

The genetic modification identified in the transgenics presented in this application can be used as a predictor of comparable modifications in the natural population with the predicted changes in biomass as described herein. Thus, the transgenics can be used to select natural variants with the described beneficial properties using techniques that are known to the skilled person and used routinely (Porth et al., 2013)

The present invention is illustrated by the following examples. It is to be understood that the particular examples, materials, amounts, and procedures are to be interpreted broadly in accordance with the scope and spirit of the invention as set forth herein.

### EXAMPLE 1

Transgenic switchgrass plants were produced using a vector including a Maize ubiquitin 1 gene promoter and intron (ZmUbi1 from Zea mays), an IRX10-L Panicum virgatum gene (Pavir.Ca01824), and a Nopaline synthase gene terminator (NOS T from Agrobacterium tumefaciens). Use of a screenable marker included a switchgrass polyubiquitin 1 gene promoter and intron (PvUbi 1 from Panicum virgatum), the red fluorescent protein reporter pporRFP from Porites porites, and the Nopaline synthase gene terminator NOS T from Agrobacterium tumefaciens. Use of a selectable marker included a Rice actin 1 gene promoter and intron (OsAct1 from Oryza sativa), the Hygromycin B phosphotransferase gene HPH from Streptomyces hygroscopicus, and a 35S gene Poly A, 35S T from Cauliflower mosaic virus.

Gene models and vector maps for IRX10-L and IRX10 knockdown lines of Populus deltiodes are shown in FIGS. 4 and 5, respectively.

Three transgenic switchgrass lines targeting Pavir.Ca01824 (previously referred to as Pavirv00004837m) were produced, 196-1, 196-3, and 196-5. The line with the greatest increased saccharification and good growth compared to the control was selected as the top line, and two other separate transgenics made via transgenesis using the same construct were selected as comparator lines. Four transgenic Populus deltoides lines targeting Potri.012G109600 were produced, AB175.1, AB175.2, AB175.4, AB175.8, and AB175.17. Three transgenic P. deltoides lines targeting Potri.001G068100 were produced, AB176.3, AB176.4, and AB176.6.

### Results

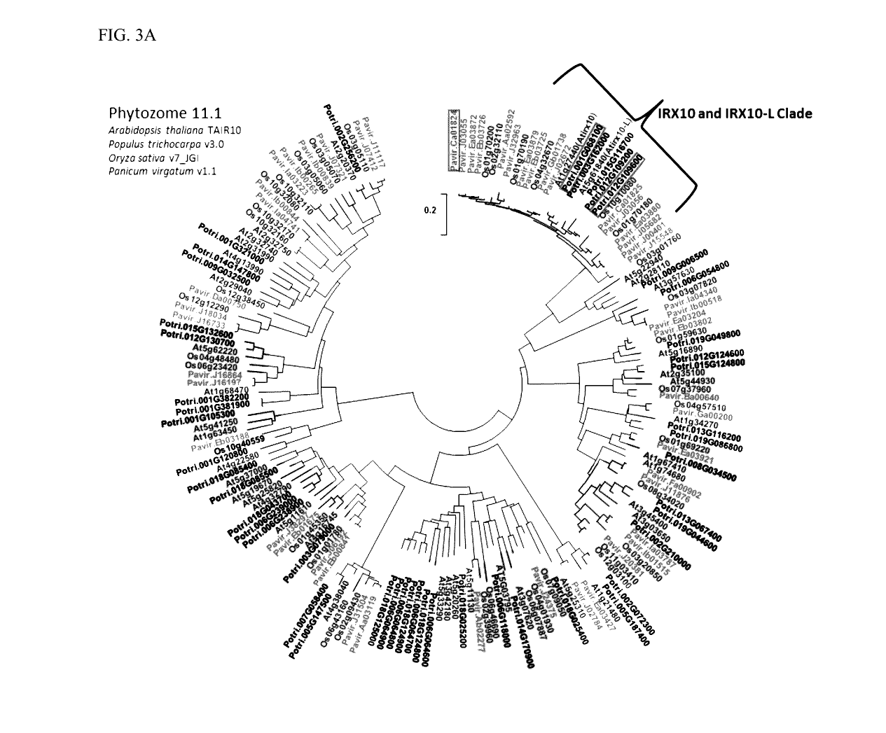

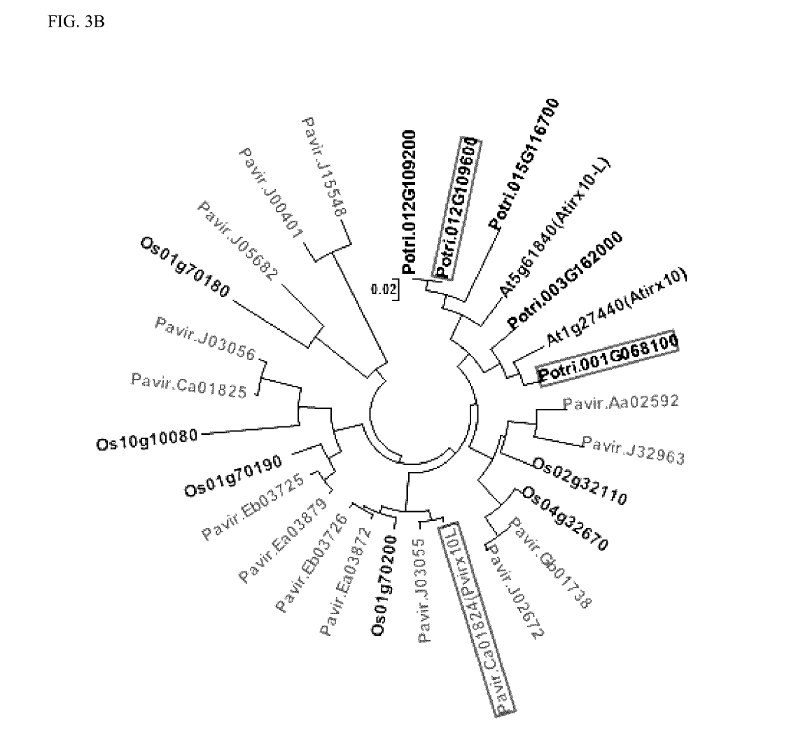

The IRX10 and IRX10-L clade of the GT47 family (FIG. 3B) has 28 members including 15 switchgrass genes. Recently, based on complementation and cell wall composition analyses, two rice genes have been characterized as IRX10 orthologs, OsIRX10 (Os1g70200) and OsGT47A (Os01g70190) (Chen et al., 2013, Mol. Plant, 6:570-573; Zhang et al., 2014, J. Plant Res., 127:423-432). The targeted switchgrass gene described here ((Pavir.Ca01824, originally referred to as Pavirv00004837m) is in the OsIRX10/OsIRX10-L subclade. Since BLAST sequence comparison with the Arabidopsis AtIRX10-L and AtIRX10 genes reveals a better match with AtIRX10-L, the targeted switchgrass gene is herein named PvIRX10-L. The IRX10-L gene targeted in P. deltoides was Potri.012G109600 and was named PtIRX10-L. The IRX10 gene targeted in P. deltoides was Potri.001G068100 and was named PtIRX10.

### Transcript Levels of Genes in Transgenic Plants.

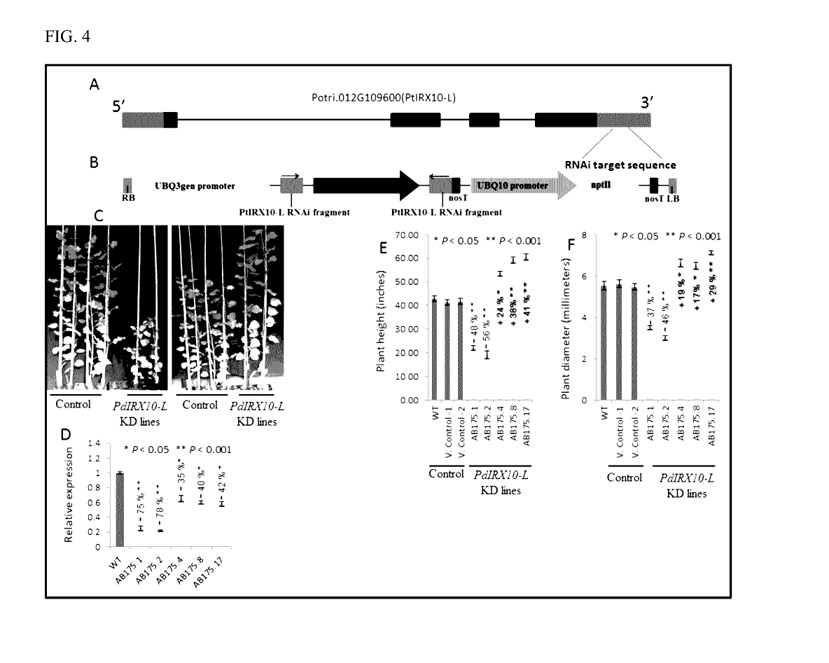

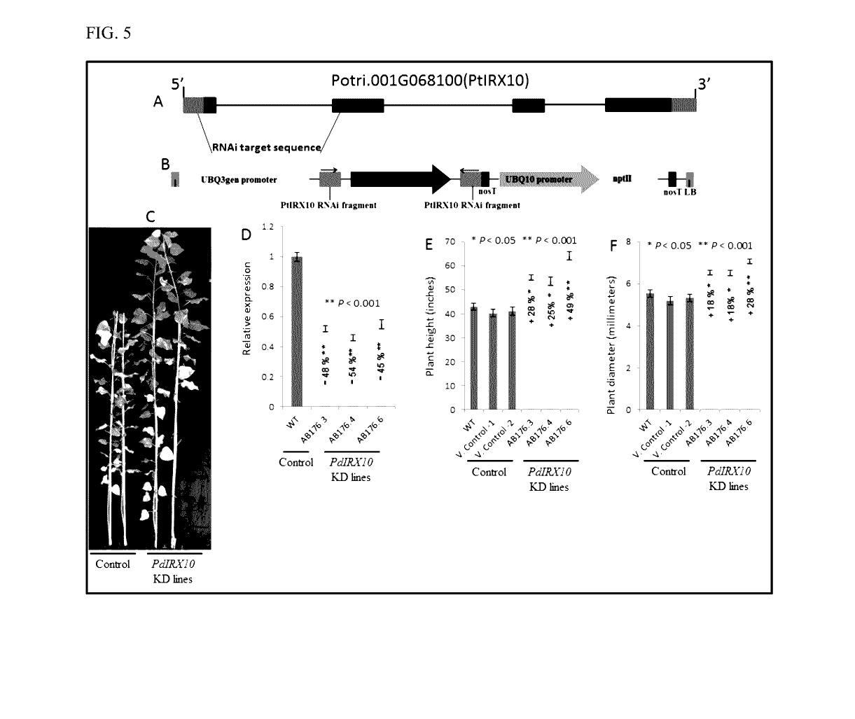

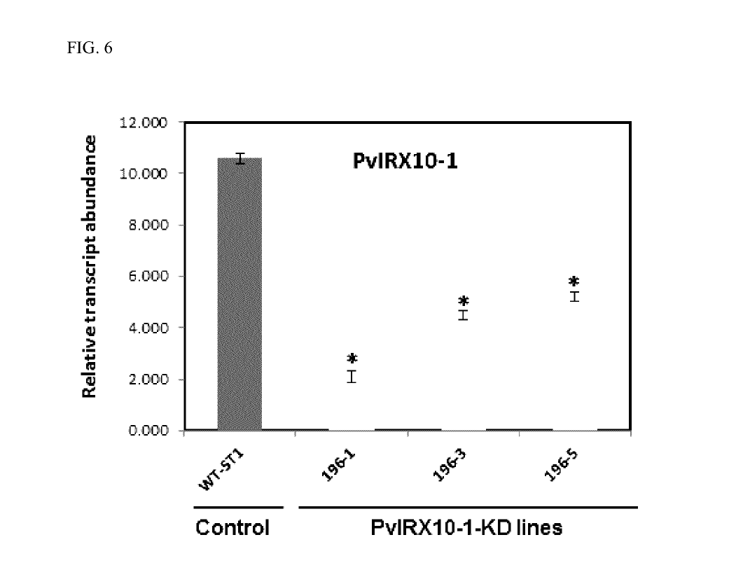

Quantitative RT-PCR analysis of the PvIRX10-L/PvIRX10-1-KD switchgrass TOP Line (196-1) revealed a 80% reduction in PvIRX10-L transcript in comparison to wild-type (WT-ST1, FIG. 6). Similarly, comparator line 196-3 had 56% and 196-5 had 51% reduction in transcript PvIRX10-L compared to wild-type. A switchgrass ubiquitin gene was used as internal standard for normalization.

Analysis of relative transcript levels of PtIRX10-L-KD lines (FIG. 4D) and the PtIRX10-KD lines (FIG. 5D) showed significant reduction in transcript levels in comparison to wild-type (WT) controls.

### Sugar Release

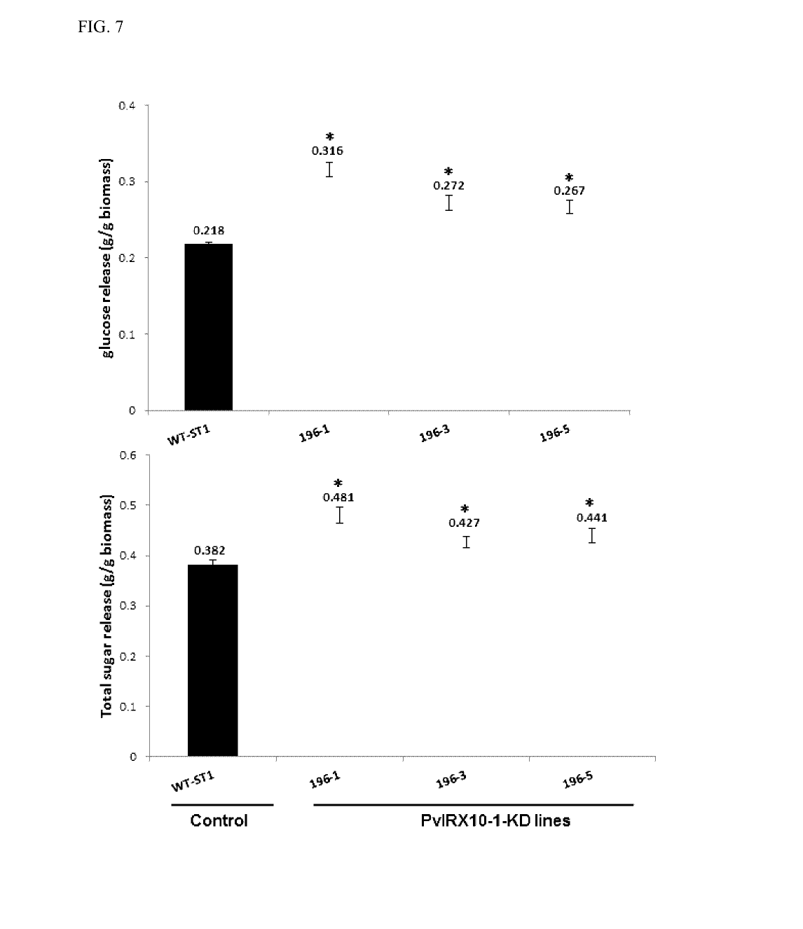

The IRX10-L knock down (KD) switchgrass (PvIRX10-L/PvIRX10-1-KD) line (196-1) had a significant 45% increase in glucose yield per gram biomass, respectively, in comparison to WT-ST1 (FIG. 7, top panel). The two comparators transgenic lines, 196-3 and 196-5, had a significant 25% and 22% increase in glucose yield per gram biomass compared to its WT counterpart (FIG. 7, top panel). Similarly, the top line (196-1) and two comparators line (196-3 and 196-5) had a significant 26%, 12% and 15% increase in glucose and total sugar yield per gram biomass, respectively, in comparison to WT-ST1 (FIG. 7, bottom panel).

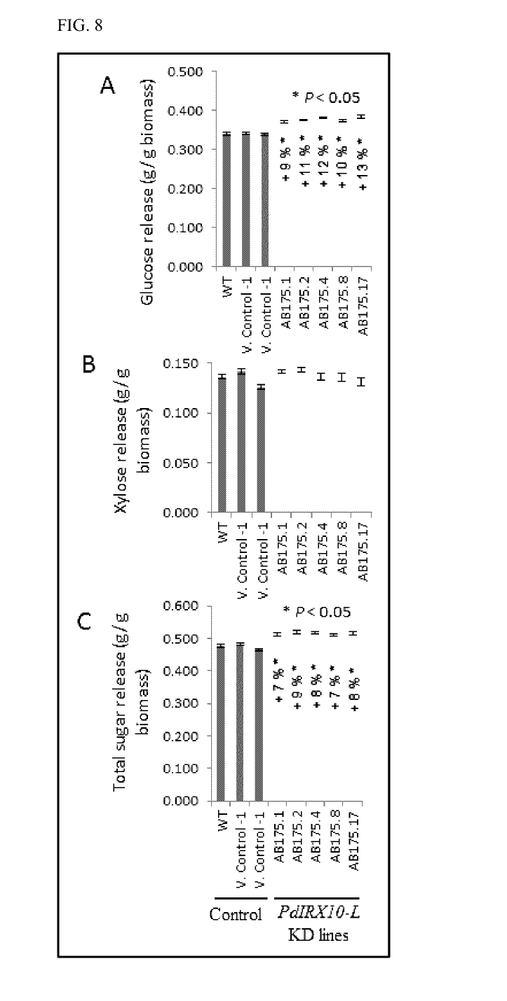

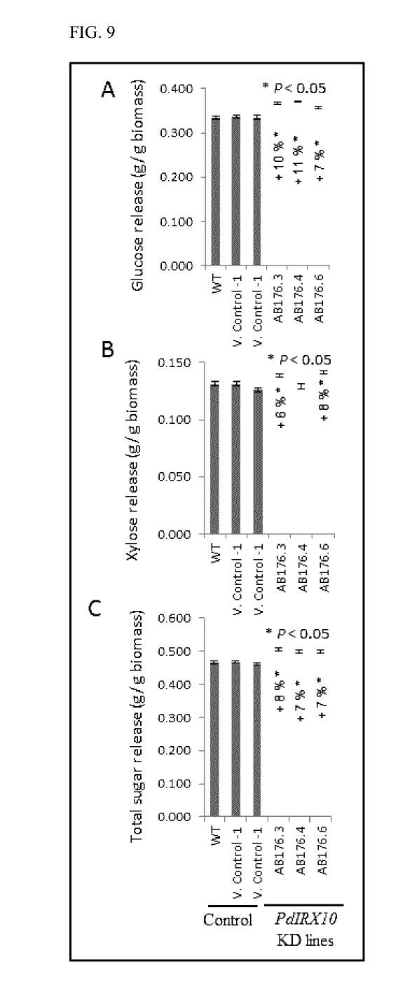

The IRX10-L and IRX10 knock down P. deltoides lines had significant increases in glucose yield per gram biomass in comparison to wild-type controls (FIG. 8, top panel, and FIG. 9, top panel, respectively). The IRX10-L and IRX10 knock down P. deltoides lines also had significant increases in total sugar yield per gram biomass in comparison to wild-type controls (FIG. 8, bottom panel, and FIG. 9, bottom panel, respectively).

### Other Phenotypes

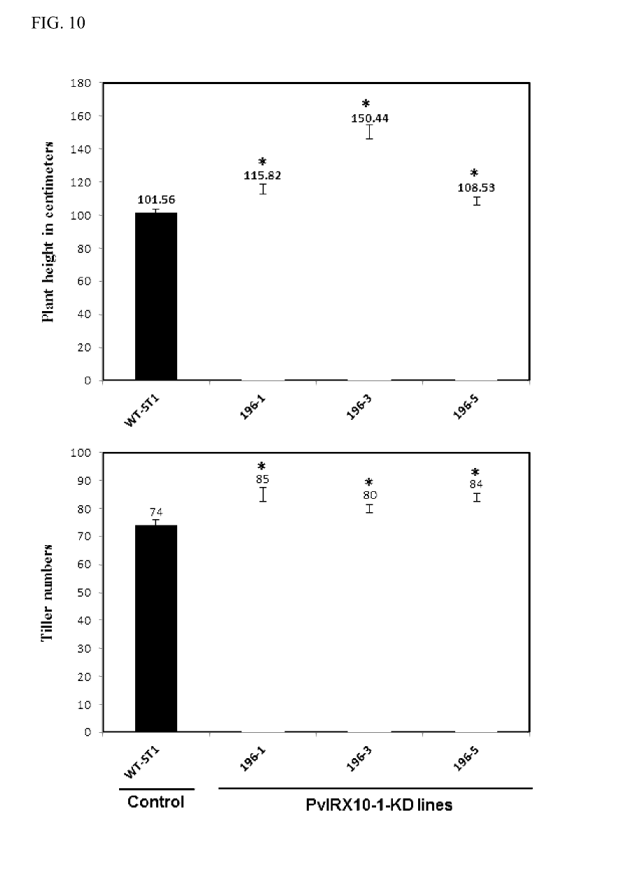

The PvIRX10-L/PvIRX10-1-KD transgenic switchgrass line 196-1 showed an increased growth phenotype. The PvIRX10-L-KD transgenic switchgrass line (196-1) had a significant 14% and 15% increased plant height and tiller number, respectively, compared to WT (FIG. 10, top panel and bottom panel, respectively). Comparator line 196-3 had 48% and 8%, and line 196-5 had 7% and 14%, increased plant height and tiller number, respectively, compared to WT (FIG. 10, top panel and bottom panel, respectively).

The PtIRX10-L-KD transgenic lines also showed an increased or decreased growth phenotype, depending upon transcript level. The two lines with the greatest reduction in transcript (AB 175.1 and AB 175.2) had reduced growth (FIG. 4C-F). The height and diameter of the transgenic knockdown lines were increased (AB175.4, AB175.8, AB175.17) (FIG. 4C-F) when the transcript level was reduced by 35-42% compared to wild type. The PtIRX10-KD lines (FIG. 5E and F), which had 48-54% reduced transcript levels compared to wild type, showed increased height and diameter compared to wild-type controls.

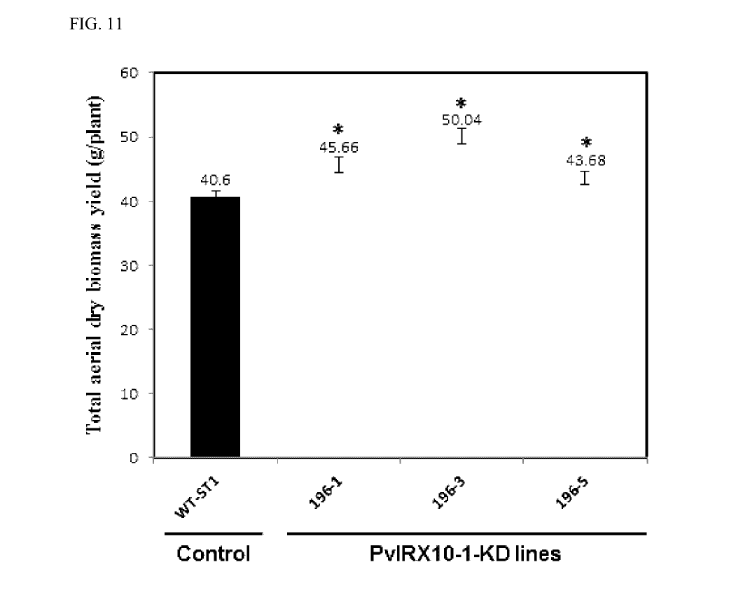

The PvIRX10-L/PvIRX10-1-KD transgenic switchgrass line 196-1 showed an increased mass yield. The PvIRX10-L-KD transgenic switchgrass line (196-1) had a 12% increase in dry weight per plant compared to the wild type ST1 (41 gm/plant) (FIG. 11). Similarly, the two comparator lines showed 23% and 8% increase in total aerial dry biomass than WT-ST1.

There is a possibility that the other genes Pavirv00024113m, Pavirv00013783m and Pavirv00048255m, especially in several subclades of IRX10 and IRX10-L clade −GT47 family, are affected by the knockdown of the PvIRX10-1/Pavirv00004837m gene. The reason is that the protein sequences of PvIRX10-1/Pavirv00004837m and Pavirv00024113m are highly conserved, exhibiting 98% amino acid sequence identity while Pavirv00013783m and Pavirv00048255m share 83% amino acid sequence identity with each other. Similarly, PvIRX10-1/Pavirv00004837m and Pavirv00013783m protein sequences have 92% amino acid identity and PvIRX10-1/Pavirv00004837m and Pavirv00048255m have 86% amino acid identity with each other, suggesting that switchgrass gene sequences in the PvIRX10 subclade of IRX10 clade −GT47 family are highly conserved.

Based on irx10/irx10-L mutant phenotypes, it has been proposed that irx10/irx10-L are responsible for the elongation of the xylan backbone (Brown et al., 2009, Plant J., 57:732-746; Wu et al., 2010, Plant Physiol., 153:542-554; Wu et al., 2009, Plant J., 57:718-731). Recent evidence supports a role of IRX10 as a catalytic subunit of a xylan synthase complex that also includes IRX9 and IRX14 (Zeng et al., 2016 Plant Physiol., 171: 93-109). Genetic analyses support the conclusion that two distinct sets of four genes each differentially contribute to glucuronoxylan biosynthesis, at least in Arabidopsis: IRX9, IRX10, IRX14 and FRA8, and IRX9-L, IRX10-L, IRX14-L, and F8H (Wu et al., 2010 Plant Physiol. 153:542-554). It has been proposed that the reducing end glycosyl residue tetrasaccharide sequence β-D-Xylp-(1-3)-α-L-Rhap-(1-2)-D-GalpA-(1-4)-D-Xylp acts as either a primer or a terminator for glucuronoxylan backbone elongation (York and O'Neill, 2008). Recently, Mortimer et al. showed using molecular genetics, in vitro assays, and expression data that IRX9L, IRX10L and IRX14 appear to be required for xylan backbone synthesis in primary cell wall-synthesizing tissues, and that although IRX9 and IRX10 are not involved in primary cell wall xylan synthesis, they are functionally exchangeable with IRX9L and IRX10-L (Mortimer et al., 2015, Plant Journal 83: 413-426). These results suggest that IRX10 is involved, in vivo, in secondary wall synthesis and that IRX10-L is involved in primary wall synthesis. Based on the observation that the synthesis of the tetrasaccharide, so-called sequence 1, at the reducing end is absent in glucuronoxylan-deficient Arabidopsis mutants irx7(fra8)/irx7-L(F8H), which is another gene of family GT47 (Brown et al., 2007, Plant J., 52:1154-1168; Zhong et al., 2005, Plant Cell 17:3390-408), it is proposed that those genes may be glycosyltransferases that synthesize sequence 1.

In regards to the IRX10/IRX10-L enzyme activity, two independent groups identified xylan xylosyltransferase activity from heterologously expressed Arabidopsis thaliana IRX10-L (Urbanowicz et al., 2014, Plant J., 80:197-206) and Arabidopsis thaliana, Plantago ovate and Physcomitrella patens IRX10 (Jensen et al., 2014, Plant J., 80(2):207-15), confirming a role for IRX10 and IRX10-L enzymes in xylan backbone synthesis. We note that the recent discovery of a cell wall proteoglycan (APAP1) that contains both xylan and pectin glycan regions could have bearing on the phenotypes caused by the knockdown expression of the switchgrass IRX10 gene described in this invention disclosure (Tan et al., 2013, Plant Cell, 25(1):270-287). For example, if the affected IRX10 synthesizes a region of xylan in a proteoglycan such as APAP1, the reduction of that structure could lead to looser walls and the associated decreased recalcitrance and increased growth.

The PvIRX10-L/PvIRX10-1-KD switchgrass lines display higher enzymatic cell wall saccharification efficiency, indicating that cellulose is more accessible in the transgenic lines. These results suggest that manipulating xylan content/xylan backbone synthesis in grass cell walls structure leads to higher saccharification efficiency in grass walls. Comparison of the IRX10/IRX-10L transcript levels in the different switchgrass and Populus IRX10/IRX10-L KD lines studied here reveals a relationship between the amount of the IRX10/IRX10-L transcript knockdown and effect on plant growth and saccharification. Increased saccharification is obtained with ˜35% or more reduction in IRX10/IRX10-L transcript compared to control. The effect of transcript reduction on growth, however is more complex. With reductions in IRX10/IRX10-L transcript of more than ˜75%, growth is negatively impacted. However, with reduction of IRX10/IRX10-L transcript of 35-55%, growth is increased.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A transgenic plant comprising decreased expression of a coding region encoding an IRX10 or IRX10-L protein compared to a control plant, wherein the transgenic plant is not SALK insertion line or a Gabi insertion line.

2. The transgenic plant of claim 1 wherein the IRX10 or IRX10-L protein is selected from a protein having at least 85% identity to SEQ ID NO:4, SEQ ID NO:6, SEQ ID NO:7, SEQ ID NO:8, SEQ ID NO:9, or SEQ ID NO:10.

3. The transgenic plant of claim 1 wherein the transgenic plant comprises a phenotype selected from decreased recalcitrance, increased growth, or the combination thereof.

4. The transgenic plant of claim 1 wherein the transgenic plant is a monocot plant.

5. The transgenic plant of claim 4 wherein the transgenic plant is a member of the family Poaceae.

6. The transgenic plant of claim 5 wherein the member of the family Poaceae is a member of a genus selected from Panicum, Zea, Oryza, Triticum, Hordeum, Sorghum, Avena, Secale, Fagopyrum, Digitara, and Chenopodium.

7. The transgenic plant of claim 4 wherein the transgenic plant is a member of the subfamily Panicoideae.

8. The transgenic plant of claim 1 wherein the transgenic plant is a woody plant.

9. The transgenic plant of claim 8 wherein the transgenic plant is a member of the genus Populus.

10. Biomass obtained from the transgenic plant of claim 1.

11. A method for using a transgenic plant, the method comprising processing a transgenic plant to result in pulp, wherein the transgenic plant comprises decreased expression of a coding region encoding an IRX10 or IRX10-L protein compared to a control plant.

12. The method of claim 11 wherein the processing comprises a physical pretreatment, a chemical pretreatment, or a combination thereof.

13. The method of claim 11 further comprising hydrolyzing the processed pulp.

14. A method for producing a metabolic product comprising:
contacting under conditions suitable for the production of a metabolic product a microbe with a composition comprising a processed pulp obtained from a transgenic plant, wherein the transgenic plant comprises decreased expression of a coding region encoding an IRX10 or IRX10-L protein compared to a control plant.

15. The method of claim 14 further comprising contacting the processed pulp with an ethanologenic microbe.

16. The method of claim 15 wherein the ethanologenic microbe is a eukaryote.

17. The method of claim 14 further comprising obtaining a metabolic product.

18. The method of claim 17 wherein the metabolic product comprises an alcohol.

19. The method of claim 18 wherein the alcohol comprises ethanol, butanol, a diol, or a combination thereof.

20. The method of claim 17 wherein the metabolic product comprises an organic acid.


SEQUENCE LISTING (10 sequences)

SEQ ID NO: 1 - protein, 412 aa, Arabidopsis thaliana
MKIHSCLSAILLFLFFSASSAKQNVRTERISGSAGDVLEDDPVGKLKVYVYELPSKYNKKLLQKDPRCLTHMFAAEIFMHRFLLSSPVRTRNPDEADWFYTPIYPTCDLTPTGLPLPFKSPRMMRSSIQLISSNWPYWNRTEGADHFFVVPHDFGACFHYQEEKAIERGILPLLQRATLVQTFGQRNHVCLDEGSITIPPFAPPQKMQAHFIPPDIPRSIFVYFRGLFYDVNNDPEGGYYARGARAAVWENFKNNPLFDISTDHPTTYYEDMQRAIFCLCPLGWAPWSPRLVEAVVFGCIPVIIADDIVLPFADAIPWEEIGVFVAEKDVPELDTILTSIPTEVILRKQRLLANPSMKRAMLFPQPAQPGDAFHQILNGLARKLPHDKSIYLKTGEKALNWTAGPVADLKPW

SEQ ID NO: 2 - DNA, 1239 bp, Arabidopsis thaliana
atgaaaatccactcttgcctctccgccattcttctgttcctcttcttctctgcttcttccgctaaacaaaatgttcggacagagcgaatttcaggaagtgctggtgatgttttggaggatgatccagtgggaaagcttaaggtttatgtatatgagcttccaagcaagtacaacaagaaattactgcaaaaggatcctcgatgtcttacacacatgtttgctgctgagatttttatgcacaggttccttttatctagtcctgttcgaacgcgtaatcctgatgaagctgattggttttacacgccaatttatcctacttgcgacctcactcctaccggcttgccattaccgtttaaatcgccgcgtatgatgagaagttcgatacaactaatctcatcaaattggccttattggaatcggactgaaggagctgatcacttctttgttgtgccccatgactttggagcttgcttccattatcaggaggaaaaagccattgaaagagggatacttccactactccagcgtgccactttggttcagacatttggtcaaaggaaccatgtctgtttggacgaaggctcgatcactattcctccttttgcacctccacagaagatgcaggcccattttattcctccagatattccacgctctatctttgtctacttccgcggtttattttatgatgttaacaacgaccccgaaggtggatattatgcaagaggtgcaagagcagcggtgtgggagaacttcaagaacaaccctttgtttgacatctcaacagatcacccgacaacatactacgaagacatgcagagagccatcttctgtctatgtcctctaggatgggctccatggagccctaggttagttgaagcagttgtgtttgggtgcattccagttatcatagcagacgacatagtactgcctttcgcagacgccatcccctgggaagaaataggagtctttgttgcggaaaaagacgtccctgaactagacacgatcctaacctcaataccaacagaagtgatcctcagaaaacagagactcctcgcaaatccttcgatgaaacgagccatgcttttccctcaacctgcacaaccaggggatgcattccatcagatacttaacgggctagctcggaagttaccccacgacaaaagcatatacttgaagacaggtgagaaggcactgaactggactgctggtccggttgctgacctgaaaccttggtaa

SEQ ID NO: 3 - protein, 415 aa, Arabidopsis thaliana
MKLSSCVLIFLLCNTFSSISAFRLSRSQPTERISGSAGDVLEDDPVGRLKVFVYELPSKYNKKILQKDPRCLNHMFAAEIYMQRFLLSSPVRTLNPEEADWFYVPVYTTCDLTPNGLPLPFKSPRMMRSAIQLIASNWPYWNRTEGADHFFVVPHDFGACFHYQEEKAIGRGILPLLQRATLVQTFGQRNHVCLKEGSITVPPYAPPQKMQSHLIPEKTPRSIFVYFRGLFYDVGNDPEGGYYARGARAAVWENFKDNPLFDISTEHPTTYYEDMQRAIFCLCPLGWAPWSPRLVEAVIFGCIPVIIADDIVLPFADAIPWEDIGVFVDEKDVPYLDTILTSIPPEVILRKQRLLANPSMKQAMLFPQPAQPGDAFHQVLNGLARKLPHERSVYLRPGEKLLNWTAGPVADLKPW

SEQ ID NO: 4 - protein, 415 aa, P. urigatum
MGKGSVRALALALAVLLACSGVAVVTAQDTERIEGSAGDVLEDNPIGRLKVFVYDLPSKYNKKLLKKDPRCLNHMFAAEIFMHRFLLSSAVRTFNPEEADWFYTPVYTTCDLTPSGLPLPFKSPRMMRSAIQLIATNWPYWNRSEGADHFFVTPHDFGACFHYQEEKAIGRGILPLLQRATLVQTFGQKNHVCLKDGSITIPPFAPPQKMQTHLIPPDTPRSIFVYFRGLFYDTGNDPEGGYYARGARASVWENFKNNPLFDISTDHPPTYYEDMQRSVFCLCPLGWAPWSPRLVEAVVFGCIPVIIADDIVLPFADAIPWEEIGMFVAEEDVPRLDSILTSIPTDVILRKQRLLANPSMKQAMLFPQPAQPGDAFHQILNGLARKLPHGDNVFLKPGERILNWTAGPPGDLKPW

SEQ ID NO: 5 - DNA, 1248 bp, P. urigatum
atggggaaggggagcgtgcgggcgctggccctggcgctcgccgtgctcctcgcctgctccggcgtcgctgttgtcacggcgcaggacaccgagcggatcgaaggaagtgctggcgatgtgctggaagataaccctattgggaggctcaaggtctttgtctatgatctccccagcaaatacaacaagaaactgctgaagaaggaccctaggtgcctgaaccacatgtttgctgctgagatattcatgcatcggttcctgttatcaagcgctgttcgaacttttaatcccgaggaagcagattggttctacacacctgtatacacaacatgcgatctgactccttcgggtcttcccttgcctttcaagtctcctcgaatgatgcgtagcgcaatccagctgattgccacaaactggccttactggaatagatcagaaggcgcagatcatttctttgtgacaccacatgactttggtgcttgctttcattatcaggaagagaaagcaattggccgaggaatcctccccttgcttcagcgtgctacgctggttcaaacctttggacagaagaaccatgtctgcctgaaggatggatccattaccataccaccatttgcgcctccccagaaaatgcaaactcaccttatacccccagatacccctcgatccatcttcgtgtacttccgcggtctgttctatgacactggcaatgatcctgaaggtggttactatgctagaggagctcgcgcatcagtctgggagaacttcaagaacaatcctctctttgacatctcgactgatcacccaccaacatactatgaagacatgcagcgttcagtgttctgcctgtgcccattgggctgggcaccatggagtcctaggctagtggaagcagtggtcttcggctgcatcccggtgatcatcgcagatgacatcgtcctgccctttgcggatgccatcccatgggaggagatcggtatgttcgttgctgaggaggatgtcccgaggctggacagcatcctgacatccattcccacagatgttatactgaggaagcaacggctcctcgcgaacccatcgatgaagcaggccatgctgttcccccagcctgctcagccaggagatgcattccaccagattctaaacggacttgcacgcaagctcccacatggtgataatgtcttcttgaagcccggggagaggatcctgaactggactgctggaccaccaggcgacctgaagccttggtag

SEQ ID NO: 6 - protein, 415 aa, P. urigatum
MGKGSGRGLALALAVLLACSGVVVVTAQDTERIEGSAGDVLEDNPVGRLKVFVYDLPSKYNKKLLKKDPRCLNHMFAAEIFMHWFLLSSAVRTFNPEEADWFYTPVYTTCDLTPSGLPLPFKSPRMMRSAIQLIATNWPYWNRSEGADHFFVTPHDFGACFHYQEEKAIARGILPLLQRATLVQTFGQKNHVCLKDGSITIPPFAPPQKMQTHLIPPDTPRSIFVYFRGLFYDNGNDPEGGYYARGARASVWENFKNNPLFDISTDHPPTYYEDMQRSVFCLCPLGWAPWSPRLVEAVVFGCIPVIIADDIVLPFADAIPWEEIGVFVAEEDVPRLDSILTSIPTDVILRKQRLLANPSMKQAMLFPQPAQPGDAFHQILNGLARKLPHGDNVFLKPGERILNWTAGPPGDLKPW

SEQ ID NO: 7 - protein, 415 aa, P. urigatum
MRRRALAIAILAAVLFLGAEAQAVQQGHQTERISGSAGDVLEDNPVGRLKVYVYDLPSKYNKKLLKKDPRCLNHMFAAEIFMHRFLLSSAVRTFNPEEADWFYTPVYTTCDLTPSGLPLPFKSPRMMRSAIELIATNWPYWNRSEGADHFFVTPHDFGACFHYQEEKAIGRGILPLLQRATLVQTFGQKNHVCLKDGSITIPPFAPPQKMQAHLIPPDTPRSIFVYFRGLFYDTSNDPEGGYYARGARASVWENFKNNPLFDISTDHPPTYYEDMQRSVFCLCPLGWAPWSPRLVEAVVFGCIPVIIADDIVLPFADAIPWEEIGVFVAEEDVPKLDSILTSIPTDVILRKQRLLANPSMKQAMLFPQPAQAGDAFHQILNGLARKLPHGDNVFLKAGERVLNWTAGPPGDLKPW

SEQ ID NO: 8 - protein, 415 aa, P. urigatum
MRRWALAIAILAAVLFLGAEAQAVQQGHQTERISGSAGDVLEDNPVGRLKVYVYDLPSKYNKKLLKKDPRCLNHMFAAEIFMHRFLLSSAVRTFNPEEADWFYTPVYTTCDLTPSGLPLPFKSPRMMRSAIELIATNWPYWNRSEGADHFFVTPHDFGACFHYQEEKAIGRGILPLLQRATLVQTFGQKNHVCLKDGSITIPPFAPPQKMQAHLIPSDTPRSIFVYFRGLFYDTSNDPEGGYYARGARASVWENFKNNPLFDISTDHPPTYYEDMQRSVFCLCPLGWAPWSPRLVEAVVFGCIPVIIADDIVLPFADAIPWEEIGVFVAEEDVPKLDSILTSIPTDVILRKQRLLANPSMKQAMLFPQPAQPGDAFHQILNGLARKLPHGDNVFLKPGERVLNWTAGPPGDLKPW

SEQ ID NO: 9 - protein, 413 aa, Populus trichocarpa
MRTCLWVFAVVLVFGFVDGKKIERLRTERISGGSAGDVLDDDPVGRLKVYVYELPSKYNKKLLQKDPRCLTHMFAAEIFMHRFLLSSPVRTLNPDEADWFYSPIYPTCDLTPMGLPLPFKSPRMMRSAIQLISSNWPYWNRTEGADHFFVVPHDFGACFHYQEEKAIERGILPLLQRSTLVQTFGQRNHVCLNEGSITIPPFAPPQKMQAHQIPPDIPRSIFVYFRGLFYDVNNDPEGGYYARGARAAVWENFKNNPLFDISTDHPTTYYEDMQRAIFCLCPLGWAPWSPRLVEAVVFGCIPVIIADDIVLPFADAIPWEEIGVFVAEEDVPNLDTILTSIPPEVILRKQRLLANPSMKRAMLFPQPAQPGDAFHQILNGLARKLPHDRSVYLKSGQNILNWTAGPVGDLKPW

SEQ ID NO: 10 - protein, 417 aa, Populus trichocarpa
MEIWKWVFVGVLWYAGFVLSTGAVELGRSQHTERISGSAGDVLEDDPVGRLKVFVYELPRKYNKKILQKDPRCLTHMFAAEIFMHRFLLSSPVRTLNPDEADWFYTPVYTTCDLTTNGLPLPFKSPRMMRSAIQLISSNWPYWNRTEGADHFFVVPHDFGACFHYQEEKAIERGILPLLQRATLVQTFGQRNHVCLKDGSITVPPYAPPQKMQTHLIPEKTPRSIFVYFRGLFYDVGNDPEGGYYARGARAAVWENFKDNPLFDISTEHPATYYEDMQRAVFCLCPLGWAPWSPRLVEAVIFGCIPVIIADDIVLPFADAIPWEEIGVYVDEEDVPNLDTILTSIPPEVILRKQRLLANPSMKQAMLFPQPAQPGDAFHQVLNGLARKLPHDRSVYLRPSEKILNWTAGPVGDLKPW